(19) United States
(12) Plant Patent Application Publication
(10) Pub. No.: US 2026/0020509 P1
(43) Pub. Date: Jan. 15, 2026

(54) PETUNIA-CALIBRACHOA HYBRID PLANT NAMED ‘KLECA24829’

(50) ;

(71) Applicant: Klemm+Sohn GmbH & Co. KG, Stuttgart (DE)

(72) Inventor: Anita STÖVER, Ostfildern (DE)

(73) Assignee: Klemm+Sohn GmbH & Co. KG, Stuttgart (DE)

(21) Appl. No.: 18/768,281

(22) Filed: Jul. 10, 2024

(51) Int. Cl. A01H 6/82; A01H 5/02

(52) U.S. Cl. CPC A01H 6/824; A01H 5/02
USPC PLT/356.11

(57) ABSTRACT

A new Petchoa plant particularly distinguished by having large violet-blue flowers and an upright habit, is disclosed.

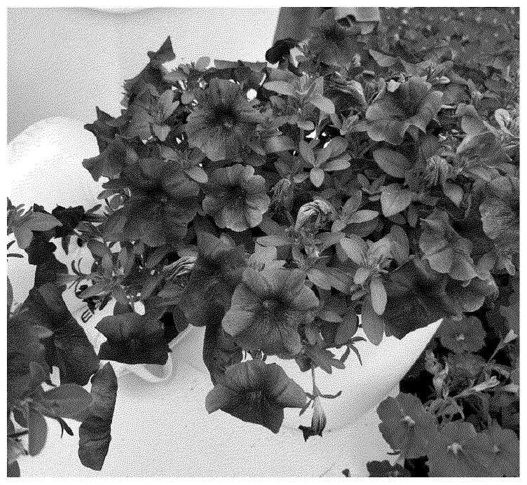

Genus and species: Petunia x Calibrachoa hybrid.

Variety denomination: ‘KLECA24829’.

### BACKGROUND OF THE NEW PLANT

The present invention comprises a new and distinct variety of Petunia x Calibrachoa hybrid, commonly known as Petchoa, and hereinafter referred to by the variety name ‘KLECA24829’. ‘KLECA24829’ originated from an open pollination of female variety SuperCal® Blue, also known as ‘SAKPXC006’, U.S. Plant Pat. No. 21,913.

In August 2016, the female variety SuperCal® Blue was open pollinated with a selection of isolated male calibrachoa varieties and seeds were obtained. The seeds were sown and plants were grown for evaluation. A plant line was selected in May 2017 and subsequently named ‘KLECA24829’. ‘KLECA24829’ was first vegetatively propagated by cuttings in Stuttgart, Germany in May 2017. ‘KLECA24829’ was found to reproduce true to type in successive generations of asexual propagation via cuttings.

### SUMMARY

The following are the most outstanding and distinguishing characteristics of this new variety when grown under normal horticultural practices in Stuttgart, Germany.
1. Large, violet-blue flowers; and
2. Upright plant habit.

### DESCRIPTION OF THE PHOTOGRAPH

This new Petchoa plant is illustrated by the accompanying photograph which shows a close-up of the whole plant.

The photograph is of an 11-week-old-plant grown outdoors in Stuttgart, Germany in June 2023. The colors shown are as true as can be reasonably obtained by conventional photographic procedures.

### DESCRIPTION OF THE NEW VARIETY

The following detailed descriptions set forth the distinctive characteristics of ‘KLECA24829’. The data which define these characteristics were collected from asexual reproductions carried out in Stuttgart, Germany. Data was collected on 8-week old plants after transplant in Stuttgart, Germany. Color references are to The R.H.S. Colour Chart of The Royal Horticultural Society of London (R.H.S.), 5^{th} edition (2007).
Classification:
   Family.—Solanaceae.
   Species.—Petunia x
   Calibrachoa hybrid.
   Common name.—Petchoa.
Parentage:
   Female.—SuperCal® Blue (also known as ‘SAKPXC006’, U.S. Plant Pat. No. 21,913).
   Male.—Calibrachoa sp.
Plant description:
   Life cycle.—Annual.
   Form.—Short lived subshrub.
   Habit.—Upright.
   Height (from soil line to top of foliage).—20.0 cm.
   Spread.—30.0 cm.
Propagation:
   Type cuttings.—Tip cuttings.
   Time to produce a rooted cutting.—3 weeks.
Stems:
   Stem color.—RHS 144B.
   Pubescence.—Present.
   Pubescence color.—RHS 145C.
   Stem diameter.—0.3 cm.
   Stem length.—From soil line to first node: 0.5 cm. Total length: 20.0 cm.
   Internode length.—4.0 cm.
Leaves:
   Arrangement.—Alternate.
   Shape.—Acute elliptica.
   Apex.—Acute.
   Base.—Attenuate.
   Margin.—Entire.
   Surface texture.—Smooth.
   Surface pubescence.—Absent.
   Venation.—Pinnate.
   Venation color.—Upper surface: RHS 146C. Lower surface: RHS 146D.
   Length.—4.9 cm.
   Width.—2.2 cm.
   Color.—Upper surface: RHS 137A. Lower surface: RHS 137C.
   Fragrance.—Absent.
Flowers:
   Flowering habit.—Continuous.
   Flower type.—Single.
   Flowering requirements.—Natural sunlight.
   Duration of flowers.—7 days.
   Corolla texture (both surfaces).—Smooth.
   Corolla.—Rotate.
   Shape.—Funnel form.
   Fragrance.—Absent.
   Number of flowers per plant.—19.
Flower buds (before opening):
   Surface.—Dull.
   Length.—2.3 cm.
   Diameter.—0.5 cm.
   Shape.—Irregular to oblong.
   Color.—RHS N187C.
   Typical number per plant.—10.
Peduncle:
   Length.—1.6 cm.
   Diameter.—0.4 cm.
   Color.—RHS 145A.
   Texture.—Smooth.
   Anthocyanin color.—RHS N187B.
Flower description:
   Flower depth.—3.5 cm.
   Flower tube length.—1.5 cm.
   Flower tube diameter.—1.3 cm.
   Flower diameter.—6.2 cm.
   Flower throat diameter.—1.5 cm.
   Pedicel color.—RHS 145A.
   Calyx.—RHS 146C.
Sepals:
   Quantity.—5.
   Shape.—l Lanceolate.
   Texture.—Smooth.
   Apex.—Acute.
   Margin.—Entire.
   Length.—1.6 cm.
   Diameter.—0.3 cm.
   Sepal color.—Upper surface: RHS N137A. Lower surface: RHS 137C.
Petals:
   Shape.—Reniform.
   Quantity.—5, fused.
   Length.—3.2 cm.
   Apex.—Obtuse.
   Margin.—Entire.
   Base.—Wide open, fused.
   Pubescence.—Absent.
   Lobe length.—2.9 cm.
   Lobe width.—3.4 cm.
   Color.—Lobe color: Upper surface: RHS N89B. Lower surface: RHS N80A. Corolla tube color: Inner: RHS 150C. Outer: RHS 150C.
   Fragrance.—Absent.
   Lobing intensity.—Moderate.
   Vein intensity.—Low.
Reproductive organs:
   Stamen.—Quantity: 5. Anther color: RHS 9A. Anther length: 0.2 cm. Filament length: 0.9 cm. Filament color: RHS 8D. Pollen amount: Very sparse. Pollen color: RHS 9A.
   Pistil.—Quantity: 1. Length: 1.6 cm. Ovary: Superior. Placenta arrangement: Free, central. Stigma color: RHS 144A. Stigma shape: Rounded. Style length: 1.3 cm. Style color: RHS 145B.
Fruit/seed set: None.
Disease/insect resistance: None observed.

### COMPARISON WITH PARENTAL LINE AND KNOWN VARIETY

‘KLECA24829’ is distinguished from its female parent as described in Table 1:

TABLE 1
Comparison with Female Parental Line

| | Variety | Female parent |
|---|---|---|
| Trait | ‘KLECA24829’ | SuperCal ® Blue |
| Flower size | 6 cm in diameter | 5 cm in diameter |

‘KLECA24829’ is most similar to SuperCal® Cherry Improved (also known as ‘SAKPXC012’, U.S. Plant Pat. No. 26,332). Differences between the two varieties are described in Table 2:

TABLE 2
Comparison with Similar Variety

| | Variety | SuperCal ® Cherry |
|---|---|---|
| Trait | ‘KLECA24829’ | Improved |
| Flower color | Violet-blue | Red-purple |
| Growth habit | upright | Semi-upright |

What is claimed is:

1. A new and distinct Petunia-Calibrachoa hybrid plant named ‘KLECA24829’ as shown and described herein.
